(19) United States
(12) Patent Application Publication
(10) Pub. No.: US 2021/0268041 A1
(43) Pub. Date: Sep. 2, 2021

(54) NON-VIABLE BIFIDOBACTERIUM BIFIDUM BACTERIA AND USES THEREOF

(71) Applicant: Synformulas GmbH, Grafelfing (DE)

(72) Inventor: Clemens FISCHER, Munchen (DE)

(73) Assignee: Synformulas GmbH, Grafelfing (DE)

(21) Appl. No.: 17/253,320

(22) PCT Filed: Jun. 21, 2019
(86) PCT No.: PCT/EP2019/066448

§ 371 Date: Dec. 17, 2020

(30) Foreign Application Priority Data
Jun. 22, 2018 (EP) 18179382.9

(51) Int. Cl. A61K 35/745; C12N 1/36; A61P 1/00; A23L 33/135; A23K 10/16

(52) U.S. Cl. CPC A61K 35/745; C12N 1/36; C12R 2001/01; A23L 33/135; A23K 10/16; A61P 1/00

(57) ABSTRACT

The present invention relates to non-viable bacteria of the Bifidobacterium bifidum strain SYN-HI-001 deposited under deposit No. DSM 24514, or one or more fragments thereof, for use in therapy, particularly for use in treating a gastrointestinal disorder, such as irritable bowel syndrome. Furthermore, the present invention relates to a composition comprising, as an active ingredient, said non-viable bacteria for use in therapy, particularly for use in treating a gastrointestinal disorder. Also, the present invention relates to a method of preparing non-viable bacteria of the Bifidobacterium bifidum strain SYN-HI-001 deposited under deposit No. DSM 24514, or one or more fragments thereof, and to bacteria obtained by the inventive method for use in therapy, particularly for use in treating a gastrointestinal disorder.

The present invention relates to non-viable bacteria of the Bifidobacterium bifidum strain SYN-HI-001 deposited under deposit No. DSM 24514, or one or more fragments thereof, for use in therapy, particularly for use in treating a gastrointestinal disorder, such as irritable bowel syndrome. Furthermore, the present invention relates to a composition comprising, as an active ingredient, said non-viable bacteria for use in therapy, particularly for use in treating a gastrointestinal disorder. Also, the present invention relates to a method of preparing non-viable bacteria of the Bifidobacterium bifidum strain SYN-HI-001 deposited under deposit No. DSM 24514, or one or more fragments thereof, and to bacteria obtained by the inventive method for use in therapy, particularly for use in treating a gastrointestinal disorder.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Gastrointestinal disorders are typically correlated with a functional impairment of the gastrointestinal tract and/or correlated with undesirable gastrointestinal inflammatory activity and have a widespread prevalence. Irritable bowel syndrome (IBS) (EMA/CHMP/60337/2013), for example, is one of the most common of these disorders, with an estimated prevalence of up to 20% in the European population. IBS is characterized by recurrent episodes of functional gastrointestinal symptoms for which no organic disease is verifiable. Common IBS symptoms include abdominal pain, flatulence, bloating, diarrhoea and/or constipation. IBS patients suffer from a distinct impairment in their quality of life, which has been found to be even worse than in patients with other chronic diseases (Chang et al., 2004).

In accordance with recommendations of the European Medicines Agency (EMA), IBS can be diagnosed by the Rome III criteria which are currently widely accepted as the scientific standard for defining IBS. The current Rome III criteria define the IBS population as: abdominal pain or discomfort at least 3 days per month during the last 3 months with symptom onset at least 6 months ago associated with at least 2 criteria of (1) improvement with defecation, (2) onset associated with a change in stool frequency or (3) in stool form (EMA/CHMP/60337/2013; Rome, 2006).

The etiology leading to the diverse symptom profile in IBS patients is still incompletely understood and, due to the heterogeneous pathology of the IBS population, an efficient standard therapy is still lacking. At present, treatment of IBS mainly focuses on the patient's predominant symptoms. However, the efficacy of most medications is modest and high quality evidence is often lacking (Camilleri and Ford, 2017).

Colonic biopsy studies consistently show that the intestinal barrier function is altered in IBS patients, with significantly higher permeability seen in comparison to healthy subjects (Piche et al., 2009; Vivinus-Nébot et al., 2012). One hypothesis is, thus, that due to this increase in intestinal barrier permeability, translocation of facultative pathogenic bacteria occurs, in turn leading to IBS symptoms such as abdominal pain, urgency, constipation and diarrhoea. To enhance mucus barrier activity and to restore the gut barrier, the use of mucosal barrier protectors, such as gelatin tannate, has been suggested as a potential approach to re-establish the physiological intestinal homeostasis (Lopetuso et al., 2015).

In vitro studies have shown that specific probiotic strains are able to strengthen the intestinal barrier function providing an explanation why certain bacteria are helpful in the treatment of IBS (Resta-Lenert et al., 2006; Eun et al., 2011). Based on these findings, probiotics are becoming increasingly important in the treatment of IBS. EP-B1-2481299 describes a specific strain of Bifidobacterium bifidum which, when formulated into a probiotic formulation, was found to significantly improve the symptoms of IBS: abdominal pain/discomfort, distension/bloating, urgency and digestive disorder.

However, the mode of action and efficacy of various strains is highly strain-specific and even closely related strains may differ significantly in their effectiveness (Brenner and Chey, 2009; Layer et al., 2011). This strain specificity has been demonstrated in studies on the efficacy of Lactobacillus plantarum MF 1928 and Lactobacillus plantarum LP299V on IBS symptoms. Whereas the strain L. plantarum LP299V provided a significant improvement in IBS symptoms (Ducrotté et al., 2012), the closely related strain L. plantarum MF 1298 led to a significant worsening of symptoms in IBS patients (Farup et al., 2012). Thus, despite their close relationship, the two strains differ significantly in their effectiveness. Furthermore, numerous other strains were found to be not effective at all in the treatment of irritable bowel syndrome (Bausserman and Michail, 2005; Niv et al., 2005; Kim et al., 2005; Sen et al., 2002). Moreover, although the use of probiotic bacteria to enhance gastrointestinal health has been proposed for many years and is generally recognized as safe, it has been shown that patients with serious diseases may be at greater risk for severe complications by oral administration of probiotic bacteria. Besselink et al., 2008 reported a clinical trial with patients with predicted severe acute pancreatitis that revealed that a multispecies preparation of viable probiotics used as prophylaxis was associated with an increased mortality risk compared to placebo.

Thus, despite the fact that a lot of effort is currently being invested into obtaining a more thorough understanding of gastrointestinal disorders and into developing appropriate therapeutic approaches, there is still a need to provide efficient and safe alternatives to the currently available approaches.

This need is addressed by the provision of the embodiments characterized in the claims.

Accordingly, the present invention relates to non-viable bacteria of the Bifidobacterium bifidum strain SYN-HI-001 deposited under deposit No. DSM 24514, or one or more fragments thereof, for use in therapy (i.e. for use as a medicament/for use in treating a disorder). In particular, the invention also relates to these non-viable bacteria, or one or more fragments thereof, for use in treating gastrointestinal disorders, such as IBS.

The invention further relates to a composition comprising said non-viable bacteria, or one or more fragments thereof, for use in therapy, particularly for use in treating a gastrointestinal disorder, such as IBS. In particular, the invention refers to a composition comprising, as an active ingredient, said non-viable bacteria, or one or more fragments thereof, for use in therapy, preferably for use in treating a gastrointestinal disorder (such as IBS). Furthermore, the present invention relates to a method of preparing non-viable bacteria of the Bifidobacterium bifidum strain SYN-HI-001 deposited under deposit No. DSM 24514, or one or more fragments thereof, and to bacteria, or one or more fragments thereof, obtained by the inventive method for use in therapy, particularly for use in treating a gastrointestinal disorder, such as IBS. The invention also relates to a method of treating a gastrointestinal disorder, such as IBS, wherein the method comprises administering non-viable bacteria of the Bifidobacterium bifidum strain SYN-HI-001 deposited under deposit No. DSM 24514, or one or more fragments thereof, to a subject in need thereof. Furthermore, the present invention also relates to the use of non-viable bacteria of the Bifidobacterium bifidum strain SYN-HI-001 deposited under deposit No. DSM 24514, or one or more fragments thereof, for the manufacture of a medicament for treating a gastrointestinal disorder, such as IBS.

In accordance with the present invention, the non-viable bacteria are Bifidobacterium bifidum bacteria. Bifidobacteria belong to the family of Bifidobacteriaceae of Actinobacteria and represent a genus of gram-positive, non-motile, often branched anaerobic bacteria. They are ubiquitous inhabitants of the gastrointestinal tract, vagina and mouth of mammals, including humans. Moreover, they are one of the major genera of bacteria that make up the colon flora in mammals. The specific Bifidobacterium bifidum strain SYN-HI-001 was deposited at the Leibniz-Institute DSMZ (Deutsche Sammlung von Mikroorganismen und Zellkulturen GmbH (DSMZ), Inhoffenstr. 7B, 38124 Braunschweig, Germany) on 26 Jan. 2011 under deposit No. DSM 24514.

In accordance with the present invention, the bacteria can be in any of their life cycle forms, for example, vegetative, or stationary, provided that they are non-viable.

The term “non-viable bacteria”, as used herein, relates to bacteria that have been inactivated, i.e. which are dead. Non-viable bacteria have no metabolism and are no longer capable of replicating. Whether bacteria are capable of replicating can be determined by the skilled person without further ado, for example by plating the bacteria on agar plates and analyzing whether they are capable to grow and form colonies on said plates under conditions suitable to enable viable bacteria of said strain to grow and form colonies. The lack of colony-formation under these conditions indicates that the bacteria are non-viable.

In accordance with the present invention, the term “non-viable bacteria” includes both intact non-viable bacteria as well as bacteria that are no longer intact. Non-limiting examples of non-viable bacteria that are no longer intact include, without being limiting, bacteria that show a disruption of the bacterial cell wall.

The present invention further relates to “one or more fragments” of these non-viable bacteria of the Bifidobacterium bifidum strain SYN-HI-001. Such (a) fragment(s) can, for example, include the majority of molecules that make up the bacteria of the Bifidobacterium bifidum strain SYN-HI-001, but can also be limited to specific components of said bacteria, such as e.g. the cell wall and/or the cell membrane of the bacteria. Preferably, the fragment(s) contain(s) at least one or more of the molecules of the cell wall of the bacteria of the Bifidobacterium bifidum strain SYN-HI-001. More preferably, the fragment(s) contain(s) at least 60%, such as e.g. at least 70%, more preferably at least 80%, such as e.g. at least 90%, more preferably at least 95%, such as e.g. at least 98% and most preferably at least 99% of the cell wall molecules present in the bacteria of the Bifidobacterium bifidum strain SYN-HI-001.

In accordance with the present invention, the non-viable bacteria, or the one or more fragments thereof, are provided for use in therapy (i.e. for use as a medicament). To this end, they can be provided in any suitable form that allows them to be administered to a patient and to unfold their therapeutic potential. For example, the non-viable bacteria or the one or more fragment thereof can be formulated into a composition as detailed herein below, including a pharmaceutical, a cosmetic, a prebiotic, or a food composition.

In the context of the present invention, it was surprisingly found that heat inactivated B. bifidum SYN-HI-001 bacteria effectively improved IBS symptoms. As is shown in the appended examples, a randomized, double-blind, multi-centre and placebo-controlled study was carried out and provided convincing evidence for a strong beneficial effect of heat-inactivated B. bifidum SYN-HI-001 bacteria in IBS patients. With heat-inactivated B. bifidum SYN-HI-001 bacteria, a significantly higher response rate of 33.5% was achieved for the primary endpoint compared to 19.4% with placebo. The primary endpoint of the study was a composite response rate, as suggested by current guidelines of the EMA, defined as 30% improvement of abdominal pain and at least one of the three best categories for the symptom relief parameter (abdominal pain/discomfort bowel habits and other IBS-symptoms) in at least 50% of the treatment period. Additionally, heat-inactivated B. bifidum SYN-HI-001 bacteria significantly improved individual IBS symptoms at the end of treatment (“Subject global assessment of symptoms” (SGA), abdominal pain, distension/bloating, composite score 1 to 4, discomfort and pain associated with bowel movement) as well as health related quality of life. Especially the symptom relief response rate (adequate relief 3) was with 60.18% exceedingly high and highly significant in the Bifidobacteria group (P=0.0009).

To the inventors' best knowledge, up to date, no heat-inactivated bacterial strain has been shown to significantly improve IBS symptoms or other gastrointestinal diseases compared to placebo. Tsuchiya et al. (2004), for example, found that there was no improvement in the IBS symptoms pain, bowel habits and bloating, after intake of non-viable bacterial cells compared to baseline, whereas these symptoms significantly improved for viable probiotics compared to baseline and non-viable cells. Other groups studied the effectiveness of viable cells compared to non-viable cells in acute diarrhea and found that viable bacteria were important for therapeutic efficacy (Isolauri et al., 1991; Mitra et al., 1990). Thus, inactivation was repeatedly found to affect the efficacy of bacteria, an effect that might be explained by strain-specific susceptibility to the inactivation process. The study presented herein now provides the first evidence that heat-inactivated bacteria of the strain B. bifidum SYN-HI-001 provide significant results compared to placebo in IBS patients. Accordingly, the present invention provides for the first time a non-viable bacterial strain with a proven in vivo efficacy in the treatment of IBS. Different from established probiotic compositions for treatment of gastrointestinal disorders, which are based on viable bacteria, these non-viable bacteria of the present invention additionally provide a reduced risk of being associated with adverse side effects, which have previously been reported e.g. in Besselink et al., 2008 for seriously ill subjects. Thus, the non-viable bacteria of the present invention, as well as fragments thereof, provide a promising and safe new tool for the treatment of gastrointestinal disorders.

As explained above, the present invention particularly relates to non-viable bacteria of the Bifidobacterium bifidum strain SYN-HI-001 deposited under deposit No. DSM 24514, or one or more fragments thereof, for use in treating a gastrointestinal disorder. As the presence of a gastrointestinal disorder in a subject is often associated with impairment in the quality of life for said subject, the present invention also relates to the use of said non-viable bacteria, or the fragment(s) thereof, for improving an impairment in the quality of life caused by or associated with a gastrointestinal disorder.

The “gastrointestinal disorder” to be treated in accordance with the present invention is preferably selected from irritable bowel syndrome (IBS), inflammatory bowel disease (IBD), Crohn's disease, ulcerative colitis, pouchitis, post infection colitis, diarrhoea (including Chlostridium difficile associated diarrhoea), constipation, dyspepsia and/or associated dyspeptic symptoms, gastroparesis, intestinal pseudo-obstruction, obstructed defecation, abdominal bloating, abdominal distension, fecal impaction, abdominal pain, abdominal discomfort, pain associated with bowel movement, reduced/increased number of bowel movements and loose stool form. More preferably, the disorder is selected from IBS, IBD, Crohn's disease, ulcerative colitis, pouchitis, post infection colitis, diarrhoea (including Chlostridium difficile associated diarrhoea), constipation, dyspepsia and/or associated dyspeptic symptoms, gastroparesis and intestinal pseudo-obstruction. Even more preferably, the gastrointestinal disorder is selected from IBS, IBD, Crohn's disease, ulcerative colitis, pouchitis, post infection colitis and diarrheal disease. Most preferably, the disorder is IBS.

The present invention further relates to a composition comprising the non-viable bacteria of the Bifidobacterium bifidum strain SYN-HI-001 deposited under deposit No. DSM 24514, or one or more fragments thereof. The term “composition”, as used in accordance with the present invention, relates to a composition which comprises, as an active ingredient, at least the non-viable bacteria of the invention, or one or more fragments thereof. It is particularly preferred that at least 85%, more preferably at least 90%, such as at least 95%, even more preferably at least 98% and even more preferably at least 99% of all Bifidobacterium bifidum bacteria comprised in the composition are non-viable bacteria of the Bifidobacterium bifidum strain SYN-HI-001 deposited under deposit No. DSM 24514. It is particularly preferred that at least 99.5%, more preferably at least 99.9% and most preferably, 100% of all Bifidobacterium bifidum bacteria comprised in the composition are non-viable bacteria of the Bifidobacterium bifidum strain SYN-HI-001 deposited under deposit No. DSM 24514.

The composition may, optionally, comprise further molecules. Such further molecules can be active ingredients themselves, or can be inert, and can be included to fulfil diverse functions, such as e.g. for the stabilisation of the non-viable bacteria of the invention, or of the one or more fragments thereof, or for delaying, modulating and/or activating their function, or as a compound that provides an additional (health) benefit to the patient provided with said composition. Non-limiting examples of such further molecules include pharmaceutically acceptable and/or ingestible carriers, adjuvants, other non-viable bacterial components, further pharmacologically active agents, proteins and/or peptides, in particular proteins and/or peptides that are rich in glutamine/glutamate, lipids, carbohydrates, vitamins, minerals and/or trace elements.

The term “pharmaceutically acceptable carrier” relates to a non-toxic solid, semisolid or liquid filler, diluent, encapsulating material or formulation auxiliary of any type. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG. Particularly preferred carriers in accordance with the present invention include phosphate buffered saline solutions, water, emulsions, such as oil/water emulsions, various types of wetting agents, sterile solutions, and organic solvents, including DMSO.

The term “ingestible carrier”, as used herein, relates to a carrier that is suitable for the oral administration of the composition and that helps ingesting the ingredients of the composition. Appropriate carriers can be chosen by the skilled person without further ado, depending on the form in which the composition is to be administered. To provide some non-limiting examples: cellulose may be used as a carrier material for tablet or capsule forms; maltodextrin may be used for powder forms or milk products may be used for administration in the form of a food product, as detailed herein below.

The term “adjuvant”, as used herein, relates to a compound that modifies the effect of other compounds, such as e.g. the non-viable bacterial of the invention, or the one or more fragments thereof, while having few—if any—direct effects when given alone. Adjuvants are often added to promote an earlier, more potent response, and/or more persistent response to the active ingredient, thereby allowing for the use of a lower dosage. Non-limiting examples of adjuvants include e.g. aluminum hydroxide and aluminium phosphate, the organic compound Squalene but also compounds currently being tested or already qualified as adjuvants, such as e.g. QS21, Aluminum hydroxide and it's derivates, oil immersions, Lipid A and it's derivates (e.g. monophosphoryl lipid A (MPL), CpG motivs, Muramyldipeptid (MDP), Freund's Complete Adjuvant (FCA), Freund's incomplete Adjuvant (FIA) or MF59C (see e.g. Gargon and Van Mechelen. Recent clinical experience with vaccines using MPL- and QS-21-containing adjuvant systems. Expert Rev Vaccines. 2011 April; 10(4):471-86; Alving C R. Lipopolysaccharide, lipid A, and liposomes containing lipid A as immunologic adjuvants. Immunobiology. 1993 April; 187(3-5):430-46; Petrovsky and Aguilar. (2004). “Vaccine adjuvants: current state and future trends”. Immunol Cell Biol. 82 (5): 488-96; Weiner et al. (1997). Immunostimulatory oligodeoxynucleotides containing the CpG motif are effective as immune adjuvants in tumor antigen immunization. PNAS 94 (20): 10833-7; Yoo et al. Adjuvant activity of muramyl dipeptide derivatives to enhance immunogenicity of a hantavirus-inactivated vaccine. Vaccine. 1998 January-February; 16(2-3):216-24; Steiner et al. (1960) The local and systemic effects of Freund's adjuvant and its fractions. Archives of Pathology 70:424-434; U.S. Pat. Nos. 6,299,884 B, 6,451,325).

The term “other non-viable bacterial components”, as used herein, relates to non-viable bacteria or fragments thereof, wherein said bacteria are not the non-viable bacteria of the Bifidobacterium bifidum strain SYN-HI-001 deposited under deposit No. DSM 24514. Non-limiting examples of such non-viable bacteria include bacterial strains of the species Lactobacillus, Bifidobacterium, Saccharomyces, Streptococcus, Enterococcus and/or Bacillus.

The term “pharmacologically active agents”, as used herein, relates to chemical or biological compounds that are pharmaceutically active while being pharmaceutically tolerable to the patient. Non-limiting examples include bisacodyl, loperamide, aminosalicylate, sulfasalazine, 5-aminosalicylic acid, 4-aminosalicylic acid, benzalazine, dihydrochloride salt, olsalazine, balsalazide, and bismuth subsalicylate. In particular, the pharmacologically active agents can be selected from medicaments known to be useful in the treatment of gastrointestinal diseases. Examples of such pharmacologically active agents are known in the art and are recorded and continuously updated in pharmaceutical registers, such as e.g. the “Rote Liste” in Germany.

The term “peptide” as used herein describes a group of molecules consisting of up to 30 amino acids, whereas “proteins” consist of more than 30 amino acids. Peptides and proteins may further form dimers, trimers and higher oligomers, i.e. consisting of more than one molecule which may be identical or non-identical. The corresponding higher order structures are, consequently, termed homo- or heterodimers, homo- or heterotrimers etc. The terms “peptide” and “protein” (wherein “protein” is interchangeably used with “polypeptide”) also refer to naturally modified peptides/proteins wherein the modification is effected e.g. by glycosylation, acetylation, phosphorylation and the like. Such modifications are well-known in the art. Preferably proteins and/or peptides that are rich in glutamine/glutamate are used, as glutamine/glutamate is helpful in building up intestinal cells and in the reconstruction of damaged intestinal mucosa. Proteins and/or peptides that are rich in glutamine/glutamate include, without being limiting, milk protein, soy protein and wheat protein.

The term “lipids”, as used herein, is defined in accordance with the pertinent art. Non-limiting examples of lipids suitable as additional compounds include, without being limiting, olive oil, soy oil, rape seed oil and fish oil.

“Carbohydrates” are organic compounds consisting only of carbon, hydrogen and oxygen. Non-limiting examples of carbohydrates suitable as additional compounds include cellulose, lactose, maltodextrin, inulin, dextrose, mannitol, fructooligosaccharide, mannit, maltose, dextrin, sorbitol and fructose.

“Vitamins”, in accordance with the present invention, include water-soluble as well as water-insoluble vitamins. Non-limiting examples of vitamins suitable as additional compounds include vitamin A (e.g. retinol, retinal and carotenoids including beta carotene), vitamin B1 (thiamine), vitamin B2 (riboflavin), vitamin B3 (e.g. niacin, niacinamide, nicotinamide), vitamin B5 (pantothenic acid), vitamin B6 (e.g. pyridoxine, pyridoxamine, pyridoxal), vitamin B7 (biotin), vitamin B9 (e.g. folic acid, folinic acid), vitamin B12 (e.g. cyanocobalamin, hydroxycobalamin, methylcobalamin), vitamin C (ascorbic acid), vitamin D (e.g. ergocalciferol, cholecalciferol), vitamin E (e.g. tocopherols, tocotrienols), and vitamin K (e.g. phylloquinone, menaquinones).

Particularly preferred vitamins are the vitamins of the B group, such as vitamin B1 (thiamine), vitamin B2 (riboflavin), vitamin B3 (e.g. niacin, niacinamide, nicotinamide), vitamin B5 (pantothenic acid), vitamin B6 (e.g. pyridoxine, pyridoxamine, pyridoxal), vitamin B7 (biotin), vitamin B9 (e.g. folic acid, folinic acid), and vitamin B12 (e.g. cyanocobalamin, hydroxycobalamin, methylcobalamin).

Non-limiting examples of minerals suitable as additional compounds include magnesium, calcium, zinc, selenium, iron, copper, manganese, chromium, molybdenum, potassium, vanadium, boron, and titanium. Particularly preferred minerals are magnesium and calcium.

The term “trace element”, as used herein, relates to a chemical element that is only needed in very low quantities for the growth, development and/or physiology of the organism, preferably of a human organism. Non-limiting examples of trace elements suitable as additional compounds include iodine, copper or iron.

Suitable ratios between the non-viable bacteria or fragment(s) thereof and such further molecules can be determined by the skilled person without further ado.

For example, 15-40 g, preferably 18-35 g, more preferably 20-30 g, even more preferably 22-28 g, and most preferably 25 g of non-viable bacteria (or fragment(s) thereof) can be mixed with 50-100 g, preferably with 60-90 g, more preferably with 65-85 g, even more preferably with 70-80 g, and most preferably with 75 g of one or more carbohydrate (preferably on or more prebiotic or maltodextrin), one or more pharmaceutically acceptable compound, or mixtures thereof. Said mixture can optionally be further mixed with 30-70 g, preferably with 35-65 g, more preferably with 40-60 g, even more preferably with 45-55 g, and most preferably with 50 g of one or more pharmaceutically acceptable and/or ingestible carrier, one or more adjuvant, one or more other non-viable bacterial component, one or more further pharmacologically active agent, one or more protein and/or peptide, one or more lipid, one or more further carbohydrate, one or more vitamin, one or more mineral, one or more trace element or with a mixture of any of these molecules.

In a more preferred example, non-viable bacteria (or fragment(s) thereof) are mixed with one or more carbohydrate, preferably one or more prebiotic, in the above recited preferred amounts. Said mixture can optionally be further mixed with one or more pharmaceutically acceptable carrier, one or more further carbohydrate, one or more vitamin, one or more mineral, one or more trace element or with a mixture of any of these molecules, in the above recited preferred amounts.

In a yet more preferred embodiment, non-viable bacteria (or fragment(s) thereof) are mixed with one or more carbohydrate, preferably one or more prebiotic or maltodextrin, in the above recited preferred amounts and are further mixed with one or more pharmaceutically acceptable carrier, one or more further carbohydrate, one or more vitamin, one or more mineral, one or more trace element or with a mixture of any of these molecules, in the above recited preferred amounts.

In an even more preferred embodiment, non-viable bacteria (or fragment(s) thereof) are mixed with maltodextrin and a pharmaceutically acceptable carrier, preferably cellulose, in the above recited preferred amounts. Most preferably, non-viable bacteria (or fragment(s) thereof) are mixed with maltodextrin and cellulose at a ratio of approx. 1:3:2 (based on weight), such as e.g. 25 g of non-viable bacteria (or fragment(s) thereof) are mixed with 75 g of maltodextrin and 50 g of cellulose.

In accordance with the present invention it is particularly preferred that the composition of the invention contains the non-viable bacteria of the Bifidobacterium bifidum strain SYN-HI-001 deposited under deposit No. DSM 24514, or one or more fragments thereof, as the only active agent. It is further particularly preferred that the composition of the present invention contains no further bacterial components other than the non-viable bacteria of the Bifidobacterium bifidum strain SYN-HI-001 deposited under deposit No. DSM 24514, or one or more fragments thereof.

The composition of the invention can be any type of composition such as, for example, a pharmaceutical composition, a prebiotic composition or a food composition. It is particularly preferred that the composition is a pharmaceutical composition or a food composition.

For a prebiotic composition, the composition additionally comprises a “prebiotic compound”. The term “prebiotic compound”, as used herein, relates to a non-digestible compound that, upon ingestion by a subject, brings benefits to said subjection because of a selective stimulation of growth and/or activation of one or more beneficial bacteria that are present in the intestine of said subject. Thus, the prebiotic compound enables a rebalancing of the bacterial flora. Preferably, the prebiotic composition of the present invention comprises, in addition to the non-viable bacteria of the invention, or the one or more fragments thereof, inulin and/or a fructooligosaccharide as a prebiotic compound.

Inulin is a soluble compound that belongs chemically to the family of fructans, i.e. polysaccharides that are formed by linear chains of fructose (up to 100 fructose molecules) with a terminal glucose residue. It has been established in the art that the consumption of inulin results in an increased presence of bifidobacteria and lactobacilli in the intestine. Lactobacilli produce milk enzymes that are important for correct digestion and for the health of the colon. At the same time, a massive reduction in the number of harmful bacteria in the intestine is observed.

Fructooligosaccharides are shorter oligosaccharide fructans (up to 10 fructose molecules), which are resistant to hydrolysis by salivary and intestinal digestive enzymes. In the colon, they are fermented by anaerobic bacteria, thereby increasing the overall health of the gastrointestinal tract.

The composition of the present invention can also be in the form of a food composition. The term “food composition”, as used herein, relates to any food suitable for consumption by humans or animals. Non-limiting examples of such “food compositions” thus include e.g. animal food, e.g., extruded and pelleted animal food, or coarse mixed food as well as food for human consumption, both as solid and liquid foods, such as drinks, including drinking water and diary products, as described in more detail below. Thus, the term includes, without being limiting, a food product, a dietary supplement, or a nutrient supplement.

The term “dietary supplement”, as used herein, refers a manufactured product intended to supplement the diet of a human or animal when taken orally and is typically provided in the form of a pill, capsule, tablet, or liquid, packaged in single or multiple dose units. Dietary supplements typically do not provide significant amounts of calories, but may contain other micronutrients, such as e.g., vitamins or minerals. The term “nutritional supplement”, in accordance with the present invention, refers to a composition comprising a dietary supplement in combination with a source of calories. For example, nutritional supplements can be meal replacements or supplements, such as e.g. nutrient or energy bars, nutrient beverages or concentrates.

Preferably, the food composition is a milk product. Non-limiting examples of milk products include acidified milk, milk-based desserts, humanised milk, milk powder, milk concentrate, milk-based dressings, milk beverages as well as fermented milk products such as e.g. yoghurt, including frozen yoghurt, yoghurt preparations, such as yoghurt drinks, fermented cream, kefir, sour cream, cream fraiche, cheese and cheese spread. The term “milk product”, as used herein, includes products of either animal (i.e. dairy products) or plant origin (i.e. non-dairy products). Milk products of animal origin include products prepared from milk obtained from e.g. cow, sheep, goat or buffalo. Milk products of plant origin include fermented products of plant origin, such as products prepared from soy milk, rice milk, almond milk, coconut milk or cereal milk (e.g. milk made from oats).

In accordance with the present invention, the composition may be in solid or liquid form and can be formulated by conventional methods. For example, methods for the formulation of pharmaceutical compositions have been described in the art, e.g. in “Remington: The Science and Practice of Pharmacy”, Pharmaceutical Press, 22^{nd} edition. Generally, compositions are prepared by contacting the components of the composition uniformly and intimately with the desired additional compounds. Then, if necessary, the product is shaped into the desired formulation.

Depending on the type of composition, the compositions can be formulated in various forms. For example, pharmaceutical, as well as prebiotic compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration are particularly preferred. For oral administration, the composition is to be administered by oral ingestion, particularly by swallowing. The composition can thus be administered to pass through the mouth into the gastrointestinal tract. Non-limiting examples of preferred forms for oral administration include coated and uncoated tablets, soft gelatin capsules, hard gelatin capsules, lozenges, troches, cachet, wafer, capsule, solutions, emulsions, suspensions, syrups, elixirs, powders and granules for reconstitution, dispersible powders and granules (e.g. aseptically packed powders or granules), medicated gums, chewing tablets and effervescent tablets, which may contain flavoring or coloring agents, for immediate-, delayed-, modified-, sustained-, pulsed- or controlled-release applications. The composition may also be incorporated into food products, in order to provide a food composition.

Tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, cellulose, or high molecular weight polyethylene glycols. For aqueous suspensions and/or elixirs, the non-viable bacteria of the invention, or one or more fragments thereof, may be combined with various sweetening or flavoring agents, coloring matter or dyes, with emulsifying and/or suspending agents and with diluents such as water, ethanol, propylene glycol and glycerin, and combinations thereof.

As mentioned, the non-viable bacteria (or fragment(s) thereof) or the composition of the invention are/is for use in therapy, more specifically, for use in treating a gastrointestinal disorder. Accordingly, a therapeutically effective amount of the non-viable bacteria of the Bifidobacterium bifidum strain SYN-HI-001 deposited under deposit No. DSM 24514, or of one or more fragments thereof, is to be used in accordance with the present invention. It will be appreciated that the length of treatment needed to observe changes, and the interval following treatment for responses to occur, vary depending on the specific medical use. The particular amounts and times may be determined by conventional tests which are well known to the person skilled in the art.

For example, for a pharmaceutical composition, the dosage regimen is typically determined by the attending physician and depends on clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. A proposed, yet non-limiting dose for oral administration to a human (of approximately 70 kg body weight) may be 0.05 to 2000 mg, preferably 0.1 mg to 1000 mg, of the active ingredients per unit dose. The unit dose may be administered, e.g., 1 to 3 times per day. The unit dose may also be administered 1 to 14 times per week, e.g., with one to two administrations per day. Similar amounts and administration intervals are also appropriate for other types of compositions, such as prebiotic or food compositions.

It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian. Typically, the components of a pharmaceutical composition to be used for therapeutic administration must be sterile.

In accordance with the present invention it is particularly preferred that the non-viable bacteria are to be administered orally in a daily amount of at least about 10² non-viable cells.

More preferably, the non-viable bacteria are to be administered orally in a daily amount of at least about 10³, such as at least about 10⁴, such as at least about 10⁵, such as at least about 10⁶, such as at least about 10⁷, or such as at least about 10⁸ non-viable cells and, most preferably, in a daily amount of at least about 10⁹ non-viable cells. It is particularly preferred that an administration interval of about 24 hours, more preferably exactly 24 hours, between administrations is observed for the entire treatment period.

Further preferred is that the non-viable bacteria are to be administered orally as two units of the dosage form per day, such as for example as two tablets or two capsules per day. Preferably, each unit of a dosage form comprises at least about 5×10¹ non-viable cells, more preferably at least about 5×10², such as at least about 5×10³, such as at least about 5×10⁴, such as at least about 5×10⁵, such as at least about 5×10⁶, such as at least about 5×10⁷ non-viable cells and, most preferably, each unit of a dosage form comprises at least about 5×10⁸ non-viable cells. Thus, by administration of two units of a dosage form per day, the above described preferred total amounts of between at least about 10² to 10⁹ non-viable cells per day will be administered. It is further particularly preferred that the daily amount of the non-viable bacteria, such as e.g. the two tablets or two capsules per day, is administered at one time, and preferably always at about the same time of day, most preferably with a meal. In other words, where the total daily amount is administered in the form of two tablets or two capsules, it is preferred that both tablets/capsules are taken at the same time.

Most preferably, the gastrointestinal disorder to be treated by the above recited preferred amounts is IBS.

Preferred amounts of the prebiotic compound in accordance with the present invention include at least 1 g, preferably at least 2 g, more preferably at least 3 g, even more preferably at least 4 g, and most preferably at least 5 g of prebiotic compound per 1 g of non-viable cells and/or fragment(s) thereof, more preferably per 10⁸ non-viable cells (or fragment(s) thereof).

The non-viable bacteria (or fragment(s) thereof) or the composition of the invention can be administered in monotherapy (e.g., without concomitantly administering any further therapeutic agents, or without concomitantly administering any further therapeutic agents against the same disease that is to be treated or prevented with the non-viable bacteria (or fragment(s) thereof) or the composition of the invention). However, the non-viable bacteria (or fragment(s) thereof) or the composition of the invention can also be administered in combination with one or more further therapeutic agents. If the non-viable bacteria (or fragment(s) thereof) or the composition of the invention are/is used in combination with a second therapeutic agent active against the same disease or condition, the dose of each substance may differ from that when the corresponding substance is used alone, in particular, a lower dose of each substance may be used. The combination of the non-viable bacteria (or fragment(s) thereof) or the composition of the invention with one or more further therapeutic agents may comprise the simultaneous/concomitant administration of the non-viable bacteria (or fragment(s) thereof) or the composition of the invention and the further therapeutic agent(s) (either in a single pharmaceutical formulation or in separate pharmaceutical formulations), or the sequential/separate administration of the non-viable bacteria (or fragment(s) thereof) or the composition of the invention and the further therapeutic agent(s). If administration is sequential, either the non-viable bacteria (or fragment(s) thereof) or the composition of the invention or the one or more further therapeutic agents may be administered first. If administration is simultaneous, the one or more further therapeutic agents may be included in the same pharmaceutical formulation as the non-viable bacteria (or fragment(s) thereof) or the composition of the invention, or they may be administered in two or more different (separate) pharmaceutical formulations.

The subject or patient to be treated in accordance with the present invention may be an animal (e.g., a non human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

Further preferred is that the non-viable bacteria, or the one or more fragments thereof, or the composition are administered, or are to be administered, to an immuno-compromised subject. An “immuno-compromised” subject is any subject whose immune system is not fully functional. For example, said subject's immune system might not be able to fight infectious diseases and/or cancer. Such subjects are also referred to as having an “immunodeficiency”. A subject can be born with an immunodeficiency (primary immunodeficiency), or it can acquire an immunodeficiency (secondary immunodeficiency), for example due to HIV infection, old age, or environmental factors, such as mal-nutrition. In addition, certain drugs, such as steroids, which are often prescribed in the context of organ transplant surgery as an anti-rejection measure and in patients suffering from an overactive immune system, as in autoimmune diseases, can lead to an immunosuppression. Preferably, the immuno-compromised subject is a human. More preferably, the immuno-compromised subject is a human having an a primary immunodeficiency, a human having a secondary immunodeficiency, preferably due to HIV infection, or a human whose immune system is medically suppressed.

The present invention further relates to a method of preparing non-viable bacteria of the Bifidobacterium bifidum strain SYN-HI-001 deposited under deposit No. DSM 24514, or one or more fragments thereof, the method comprising: (a) providing bacteria of the Bifidobacterium bifidum strain deposited under deposit No. DSM 24514; and (b) inactivating the bacteria provided in step (a) to obtain non-viable bacteria of the Bifidobacterium bifidum strain SYN-HI-001, or one or more fragments thereof.

In accordance with this method of the invention, bacteria of the Bifidobacterium bifidum strain deposited under deposit No. DSM 24514 are provided in a first step. In order to provide sufficient amounts of these bacteria, they may be cultured and amplified prior to step (a) by means known in the art; e.g. as described in “Probiotics and Health Claims”, Wolfgang Kneifel, Seppo Salminen, John Wiley & Sons; 1. Edition (7 Jan. 2011). For example, viable cells of B. bifidum SYN-HI-001 can be grown in a protein-rich liquid growth medium. In general, a suitable medium for growing and/or fermenting the Bifidobacterium bifidum strain in accordance with step (a) comprises at least water, dextrose, yeast extract and minerals. The standard medium typically used is MRS broth (Difco, Detroit, Mich., USA) supplemented with 0.05% L-cysteine hydrochloride (cMRS). The skilled person is well aware of the fact that also other media may be used for growing, fermenting and pre-culture of bacterial organisms. Typically, a volume of at least 200 mL, preferably of at least 300 mL, more preferably of at least 400 mL, even more preferably of at least 500 mL and most preferably of at least 600 mL of standard medium is inoculated with at least one cell of the bacterial strain and the culture is grown over night anaerobically, e.g., at 37° C. at 220 rpm. Additional fermentation steps, such as e.g. in higher volumes may additionally be carried out.

In a second step (step (b)), the bacteria are then inactivated to obtain non-viable bacteria. Means and methods for inactivating bacteria are well known in the art and include, without being limiting, inactivation by exposure to heat, pressure, sonication, irradiation, such as e.g. by ultra-violet rays, drying, pulsed electric field (PEF), supercritical CO₂ and/or a pH change. Preferably, the inactivation is a heat inactivation. Inactivation may be performed directly in the fermentation vessel, or in a separate vessel. Step (b) can be carried out once or may be repeated as often as deemed necessary. Successful inactivation may be tested before further use of the preparation by plating an aliquot of the inactivated bacterial preparation on a culture medium and culturing under standard conditions.

After the inactivation in step (b), the resulting composition can be employed as is, or can be further processed. Further processing includes one or more additional method steps, for example for harvesting and/or purification of the non-viable bacteria or of specific fragments thereof. Means and methods for such harvesting and/or purification steps are well known in the art and include, e.g. centrifugation such as e.g. centrifugation by using a disc centrifuge or separator (e.g. as provided by GEA Westfalia Separator Group GmbH (Oelde, Germany)). The cells may then be stabilised, freeze-dried, milled and sieved using standard means and methods. All of the above methods are known in the art and have been described, e.g. in “Probiotics and Health Claims”, Wolfgang Kneifel, Seppo Salminen, John Wiley & Sons; 1. Edition (7 Jan. 2011), “Modelling microorganisms in food”, Stanley Brul, Suzanne Van Gerwen, Marcel Zwietering; 1. Edition (2007) and “Probiotic bacteria: fundamentals, therapy, and technological aspects”, J. Paulo Sousa e Silva and Ana C. Freitas; 1. Edition (2014).

The method may comprise a further step of fragmenting the non-viable bacteria of the Bifidobacterium bifidum strain SYN-HI-001 deposited under deposit No. DSM 24514 to obtain one or more fragments of the bacteria. This step can also be combined with step (b), i.e. the step of inactivation of the bacteria. For example, the inactivation in step (b) can be carried out under conditions that are sufficiently stringent and applied for a time sufficiently long to lead to the destruction of the bacteria. Such methods include, for example, any one of boiling the bacteria in e.g. an ethanol/water or a propanol/water mixture (or any other extraction solution or mixture of solvents that is suitable for extracting polar and non-polar substances), subjecting the bacteria to freeze-thaw treatment (e.g., to multiple freeze-thaw cycles), subjecting the bacteria to sonication, treating the bacteria with a detergent, such as sodium dodecyl sulfate (SDS), octylphenoxypolyethoxyethanol (e.g., Nonidet P-40 or IGEPAL CA-630), Triton X-100, n-dodecyl-β-D-maltopyranoside (DDM), digitonin, Polysorbate 20 (e.g., Tween 20), Polysorbate 80 (e.g., Tween 80), 3-[(3-cholamidopropyl)dimethylammonio]-1-propanesulfonate (CHAPS), urea, cholate, sodium lauroyl sarcosinate (sarkosyl), or any combination thereof. After the destruction of the bacteria, fragments of interest can be isolated in an additional step (c), if desired. For example, (the) cell wall (fractions) and/or the cell membrane (fractions) can be separated from the remainder of the bacterial cells, e.g. the cytoplasm and cytoplasmic components by various methods, including without being limiting filtration, preferably filtration using a filter with a pore size of about 0.5 μm to about 2 μm, more preferably of about 1 μm, and centrifugation.

In an exemplary embodiment, the following approach can be applied: viable bacteria of the Bifidobacterium bifidum strain deposited under deposit No. DSM 24514 are grown in a protein-rich liquid growth medium, heat-inactivated in a fermentation vessel, centrifuged and subsequently freeze-dried, milled and sieved. Furthermore, the dry powder inactivated bacteria can then be mixed with an excipient and filled into capsules, such as uncoated cellulose capsules, in an amount of approx. 0.5×10⁹ non-viable bacteria.

The non-viable bacteria obtained or obtainable by the method of the present invention can then be used in therapy, particularly for treating a gastrointestinal disorder, as described herein above.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if it is indicated that a component of a composition “may” be present, the invention specifically relates to both possibilities, i.e., that the corresponding component is present (contained in the composition) or that the corresponding component is absent from the composition.

As used herein, unless explicitly indicated otherwise or contradicted by context, the terms “a”, “an” and “the” are used interchangeably with “one or more” and “at least one”. Thus, for example, a composition comprising “a” specific compound, e.g. a carrier, can be interpreted as referring to a composition comprising “one or more” of said specific compound, e.g. one or more carriers.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

Unless specifically indicated otherwise, all properties and parameters referred to herein (including, e.g., any amounts/concentrations indicated in “mg/ml”, in “% (w/v)” (i.e., mg/100 μl) or in “% (v/v)”, as well as any pH values) are preferably to be determined at standard ambient temperature and pressure conditions, particularly at a temperature of 25° C. (298.15 K) and at an absolute pressure of 101.325 kPa (1 atm).

As used herein, and unless contradicted by context, the term “treatment” or “treating” (of a disease or disorder) refers to curing, alleviating, reducing or preventing one or more symptoms or clinically relevant manifestations of a disease or disorder, or to alleviating, reversing or eliminating the disease or disorder, or to preventing the onset of the disease or disorder, or to preventing, reducing or delaying the progression of the disease or disorder. For example, the “treatment” of a subject or patient in whom no symptoms or clinically relevant manifestations of the respective disease or disorder have been identified is a preventive or prophylactic treatment, whereas the “treatment” of a subject or patient in whom symptoms or clinically relevant manifestations of the respective disease or disorder have been identified may be, e.g., a curative or palliative treatment. Each one of these forms of treatment may be considered as a distinct aspect of the present invention.

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease), palliative treatment (including symptomatic relief), or prophylactic treatment (including prevention) of the disorder or disease.

Unless otherwise defined, all technical and scientific terms used herein have the same meaning as commonly understood by one of ordinary skill in the art to which this invention belongs. In case of conflict, the patent specification, including definitions, will prevail.

It is to be understood that the present invention specifically relates to each and every combination of features and embodiments described herein, including any combination of general and/or preferred features/embodiments.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims. To give a non-limiting example, the combination of claims 18, 16 and 2 is clearly and unambiguously envisaged in view of the claim structure. The same applies for example to the combination of claims 18, 16 and 12, or the combination of claims 18, 16 and 5, etc.

The figures show:

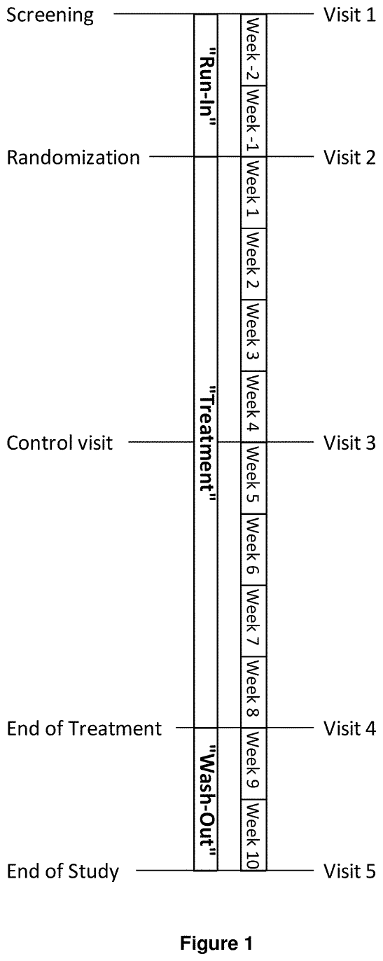

FIG. 1: Study design. A 12-week study including a run-in, treatment and wash-out period with five physician visits.

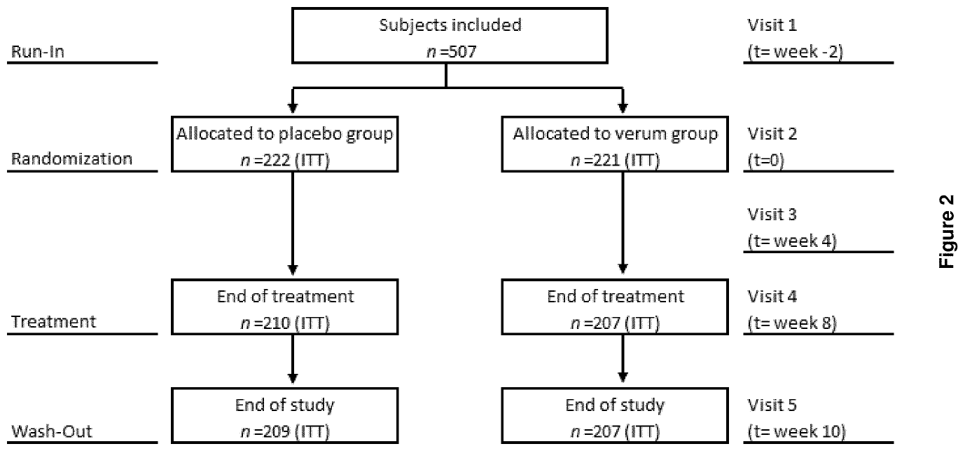

FIG. 2: Flowchart of the patients through the study. Verum=heat-inactivated B. bifidum SYN-HI-001.

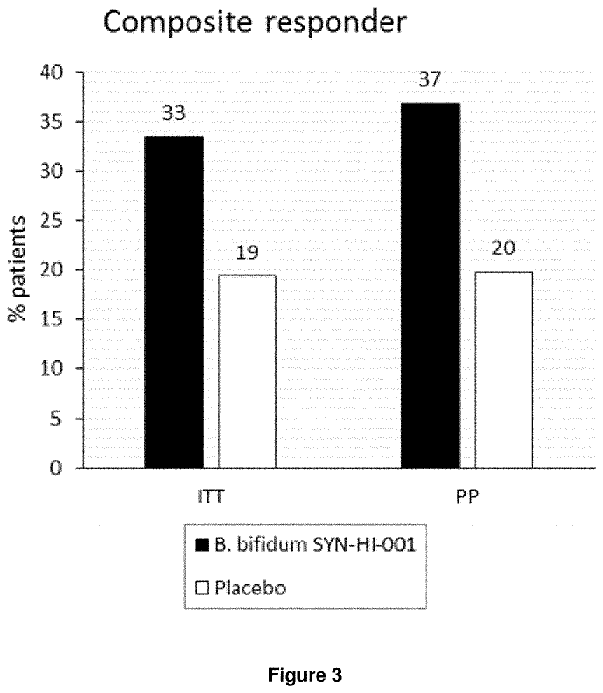

FIG. 3: Composite responders (adequate relief responder combined with pain responder) during treatment in “intention-to-treat” (ITT) and “per-protocol” (PP) population.

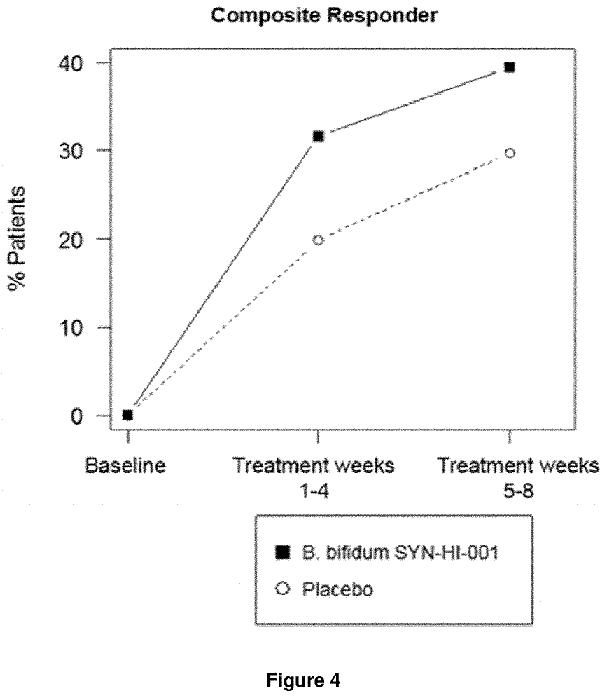

FIG. 4: Course of composite responders (adequate relief responder combined with pain responder) during both treatment intervals. Significant more responders in B. bifidum SYN-HI-001 during both treatment intervals.

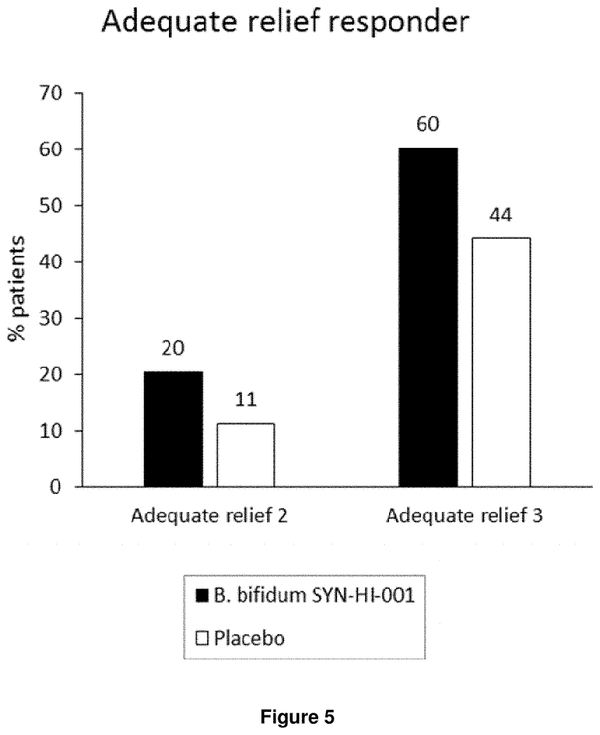

FIG. 5: Adequate relief responders during treatment.

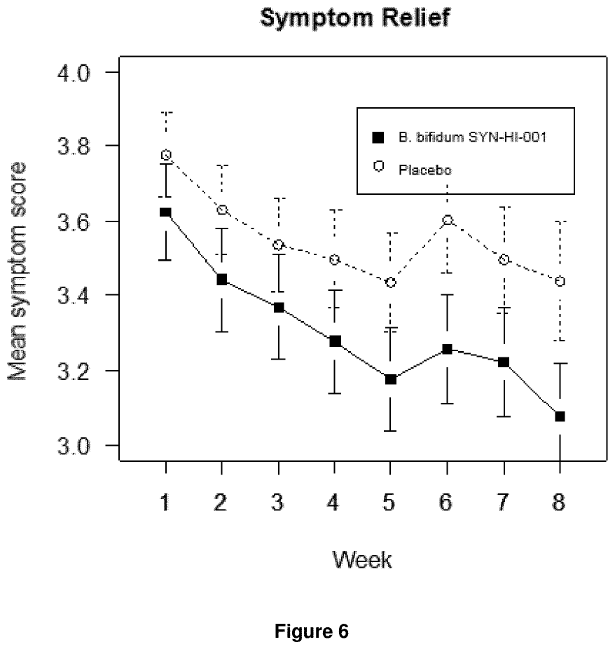

FIG. 6: Comparison of effects of B. bifidum SYN-HI-001 and placebo on symptom relief (recorded on a 1-7 scale) on a weekly basis. Significant improvement in the Bifidobacteria group vs. placebo group from the second week of treatment till the end of treatment.

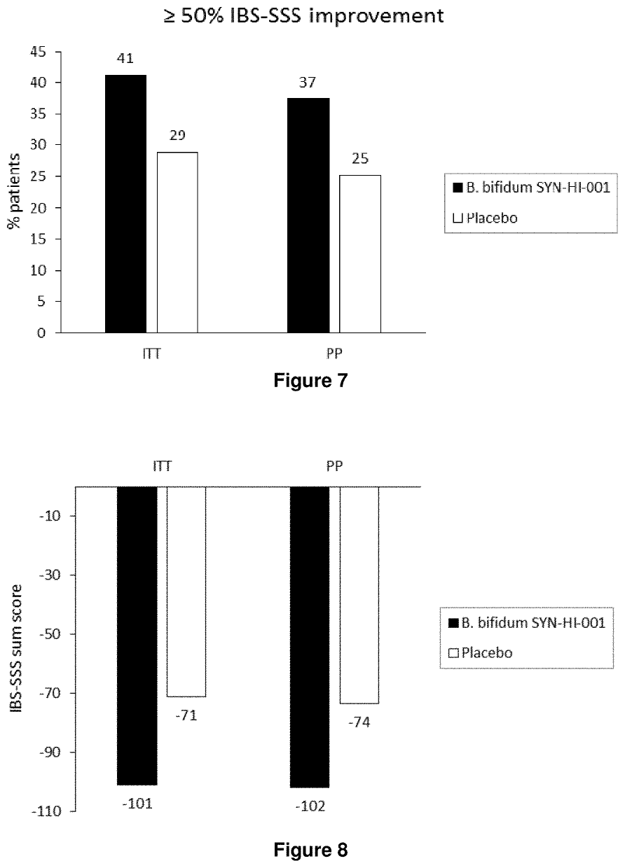

FIG. 7: Patients with ≥50% IBS-SSS improvement at the end of treatment in “intention to treat” (ITT) and “per protocol” (PP) population.

FIG. 8: Significant reduction of IB-SSS sumscore (0-500) by B. bifidum SYN-HI-001 in comparison to placebo on mean score changes from baseline to end of treatment in “intention-to-treat” (ITT) and “per-protocol” (PP) population.

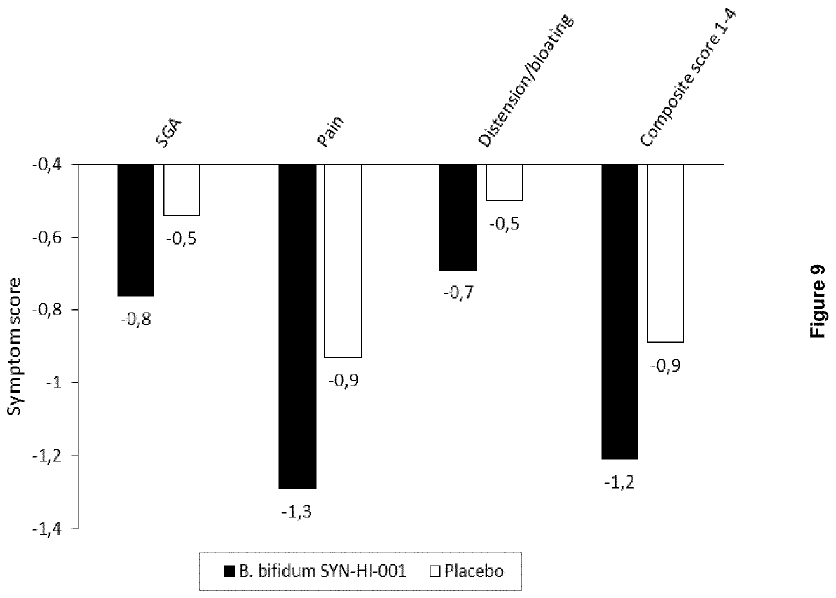

FIG. 9: Significant reduction of IBS symptoms assessed on a daily basis by B. bifidum SYN-HI-001 in comparison to placebo on mean score changes from baseline at the end of treatment (“intention-to-treat”, ITT). Composite score 1-4=arithmetic mean of four individual symptom scores (SGA, pain, distension/bloating, urgency).

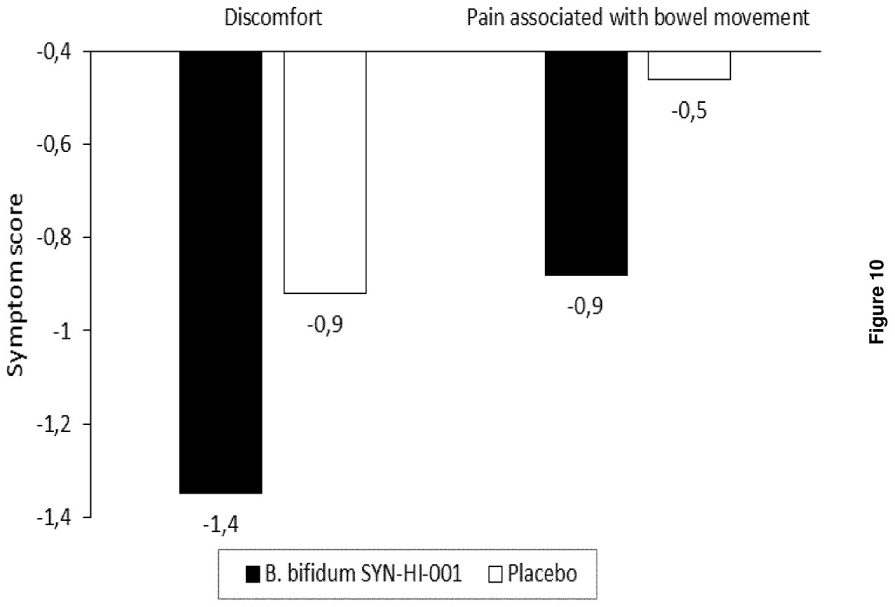

FIG. 10: Significant reduction of IBS symptoms assessed on a weekly basis by B. bifidum SYN-HI-001 in comparison to placebo on mean score changes from baseline at the end of treatment (“intention-to-treat”, ITT).

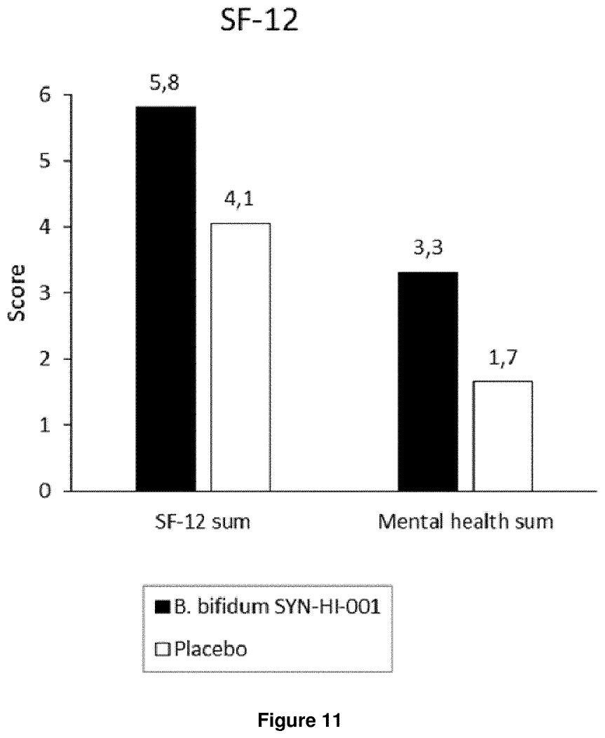

FIG. 11: Significant increase of SF-12 sum and mental health sum by B. bifidum SYN-HI-001 in comparison to placebo on mean score changes from baseline to end of treatment (“intention-to-treat”, ITT).

The following examples illustrate the invention:

### EXAMPLE 1: A DOUBLE-BLIND, MULTI-CENTRE, RANDOMIZED AND PLACEBO-CONTROLLED STUDY ASSESSING THE EFFICACY OF HEAT-INACTIVATED B. BIFIDUM SYN-HI-001 IN PATIENTS WITH IRRITABLE BOWEL SYNDROME

1.1 Study Population

Patients were recruited from principal investigators and by advertisement. The study protocol has been presented to the Ethics Committee of the Physicians Chamber of Hamburg for advisory opinion. Subjects aged between 18 and 65 years with IBS according to the Rome III criteria have been included. Excluded were individuals with inflammatory organic gastrointestinal diseases, systemic diseases, cancer, autoimmune diseases, diabetes, known lactose intolerance or immunodeficiency, abdominal surgery (except appendectomy, hernia surgery, cholecystectomy or caesarean section), no negative diagnostic result of sigmoidoscopy or colonoscopy within the last five years for patients older than 55 years, diagnosed hyperthyroidism, use of antipsychotics for at least 3 months or systemic corticosteroids during at least one month prior to study start, major psychiatric disorder, celiac disease or pregnancy.

1.2 Study Design

The study was performed as a prospective, multi-centre, randomized, double-blind, placebo-controlled, two-arm interventional study. Throughout the study, patients recorded their global IBS symptoms as well as individual IBS symptoms daily using a patient diary. Additionally, patients have been questioned at a physician site for global and individual symptoms (visit 2-5) and quality of life (visit 2-4). Physician visits were conducted at screening, after two weeks run-in phase (randomization), after 4 weeks treatment (control visit), after 8 weeks (end of treatment) and after further two weeks wash-out phase (end of study) (FIG. 1).

After patients had given their written informed consent, they qualified for the screening examination at visit 1 (day 0), which included a complete medical history and physical examination. A blood sample was taken for analysis in a central laboratory, including a pregnancy test for women. Patients were instructed to maintain their eating and life style habits throughout the study. A patient diary was handed out.

At the second visit (day 14), diaries were reviewed by the physician. Patients who had recorded at least a pain score of ≥4 on an 11-point numerical rating scale (NRS) on two days during the run-in phase and who fulfilled all further inclusion criteria and did not violate any exclusion criteria were randomized 1:1 to receive either heat-inactivated B. bifidum SYN-HI-001 or placebo. Patients were allocated to the treatment groups according to a computer-generated blocked randomization list with a block size of 4. The block size was not communicated to the investigators and the allocation was blinded to both patients and site staff. During the 8 weeks intervention period, patients consumed either two capsules comprising heat-inactivated bacteria daily or an identical appearing placebo.

In the middle of the treatment phase (visit 3, day 42), investigators discussed the course of the disease with the patient and the first part of the treatment diary was collected and reviewed. At the end of the treatment phase (visit 4, day 70), investigators reviewed and collected the remaining treatment diary.

After the treatment-free wash-out phase (visit 5, day 84), investigators collected the wash-out diary as well as unused study product and empty cans in order to confirm compliance. Furthermore, a complete physical examination was conducted and a blood sample was taken. Bisacodyl and loperamide were allowed as rescue medication. Probiotics or other medications that could influence the efficacy of the study product were not allowed.

1.3 Study Product Preparation

Viable cells of the Bifidobacterium bifidum strain deposited under deposit No. DSM 24514 were grown in a protein-rich liquid growth medium, heat-inactivated in a fermentation vessel, centrifuged and subsequently freeze-dried, milled and sieved. Furthermore, the dry powder inactivated bacteria were mixed with an excipient and filled into uncoated cellulose capsules in an amount of approx. 0.5×10⁹ cells (non-viable bacteria of Bifidobacterium bifidum strain SYN-HI-001). Placebo capsules appeared identical and contained maltodextrin.

1.4 Endpoint Definitions

The prospectively defined primary efficacy variable was as a combination of ≥30% improvement of pain on an 11-point NRS and achievement of one of the best 3 relief categories on a 7-point Likert scale with these both criteria being fulfilled in at least 4 out of 8 weeks of treatment to qualify as a treatment responder. Patients were asked to answer the daily question “If you had experienced abdominal pain during the last 24 hours, how would you rate this pain?” Possible answers ranged from 0 (no pain) to 10 (strongest imaginable pain).

Relief of symptoms was captured weekly on a 7-point Likert scale (Global Assessment of Improvement Scale). This scale was assessed every week during the 8-weeks treatments period in the patient diary. Patients were asked to answer the weekly question “Compared to the way you usually felt before taking the study medication how would you rate your relief of symptoms (abdominal pain/discomfort, bowel habits and other IBS-symptoms) during the last 7 days?” Possible answers ranged from 1 (very much relieved), 2 (considerably relieved), 3 (somewhat relieved), 4 (unchanged), 5 (somewhat worse), 6 (considerable worse) to 7 (very much worse).

Secondary efficacy variables included the “Subject global assessment of symptoms (SGA)” and individual symptoms of IBS alone, such as “abdominal pain”, “distension/bloating” and “urgency”, recorded on the same 7-point Likert scale (abdominal pain was assessed on the 11-point NRS as described above). The individual symptom scores were additionally combined into a composite symptom score as the arithmetic mean of SGA and the three individual symptom scores. Furthermore, the reduced and/or increased number of bowel movements, stool form (assessed via the Bristol Stool Form Scale), feeling of incomplete bowel evacuation and intake of other medication were reported daily in the diary and evaluated.

At the beginning, middle and end of treatment as well as at the end of the study (visits 2-5), physicians questioned the patients regarding their severity of IBS symptoms via the IBS-severity scoring system (IBS-SSS). The score is based on 5 visual analogue scales (VAS) ranging from 0 to 100 and contains for the past 10 days the severity of the symptoms “abdominal pain severity”, “abdominal pain frequency” (number of days with pain during the last 10 days), “abdominal bloating severity”, “bowel movement satisfaction” and “interference of IBS with daily activities”. Health related quality of life was assessed by the use of the SF-12 questionnaire prior to treatment, at the middle of the treatment and end of treatment (visit 2, 3 and 4).

Adverse events were recorded throughout the study and the global assessment of tolerability of the study treatment has been questioned at physician visit 3, 4 and 5. Tolerability was assessed on a 5-point scale with the question “If you consider the side effects of the study treatment, how would you rate the overall tolerability?” Possible answers ranged from 1 (very good), 2 (good), 3 (fair), 4 (not satisfactory) to 5 (bad). Vital signs were checked at every physician visit and laboratory values were examined at the screening visit and the end of study.

1.5 Statistical Methods

1.5.1 Sample Size Calculation

Sample size calculation was based on both estimated responses of placebo and treatment group for the primary endpoint and estimated differences between the IBS subgroups for main symptom scores. With 80% power, a sample size of 350 evaluable patients was calculated. With an estimated drop-out rate of 15-20% after randomization, 412 randomized patients were planned and 507 were recruited to account for possible withdrawals prior to study start.

1.5.2 Statistical Analysis

The primary objective of this study was to prove the hypothesis that the combined responder rate of adequate relief responder 3 and pain responder is significantly larger in the Bifidobacterium group compared to the placebo group. The primary endpoint was defined as a combination of ≥30% improvement of pain on an 11-point NRS (pain responder) and achievement of one of the best 3 relief categories on a 7-point Likert scale (adequate relief responder 3) and wherein these two criteria had to be fulfilled in at least 4 out of 8 weeks of treatment in order to qualify as a treatment responder. The Cochrane-Mantel-Haenszel test stratified by study center was used for the comparison of treatment arms and P<0.05 was considered as statistically significant.

The primary analysis was based on the intent-to-treat population where all successfully randomized patients were included. Weeks with missing data were automatically counted as no response. Patients who only provided baseline entries for the primary efficacy variable were considered as “non-responders” in the evaluation of the primary efficacy criterion. An additional per-protocol-analysis was performed for supportive purposes.

Secondary endpoints were analyzed descriptively based on available data. To detect treatment differences the Wilcoxon rank sum test was applied for continuous variables and the Fishers exact test for binary variables. All p values are two-sided. Secondary efficacy variables included response based on a 50% rule of symptom relief during treatment (at least improvement in 4 out of 8 weeks within the treatment period and improvement defined as at least one point reduction from baseline).

Adverse events (AE) were coded by using MedDRA using the current version at the time of study start which is used throughout the study. Coding was based on the German version and the corresponding English version was used for the final analysis. AEs were tabulated based on Preferred Terms (PT) of the MedDRA dictionary. Tabulations will include affected patients and absolute and relative frequencies of events.

All statistical analyses were performed using SAS version 9.5 for windows, SAS Institute Inc., Cary, N.C., USA.

1.6 Results

1.6.1 Subjects

From the 507 patients screened, 443 patients were successfully randomized to receive either placebo (n=222) or heat-inactivated B. bifidum SYN-HI-001 (n=221). All randomized patients were analyzed for intent-to-treat (ITT) and adverse events (n=443). A total of 377 patients (187 placebo and 190 B. bifidum SYN-HI-001) were analyzed as per-protocol (FIG. 2).

1.6.2 Baseline Characteristics

Baseline characteristics and demographics were well balanced between the two treatment groups. 24.2% were classified as constipation-predominant IBS (IBS-C), 40.0% as diarrhoea-predominant IBS (IBS-D), 7.7% as mixed IBS (IBS-M) and 28.2% as unsubtyped IBS (IBS-U) with no significant differences between the Bifidobacteria and the placebo group.

On average, patients were 41.3 years, with 69.3% females, a mean height of 171.9 cm, mean weight of 73.0 kg and a mean BMI of 24.6, with no significant differences between the two treatment groups (Table 1).

TABLE 1
Demographic characteristics of the “intention-to-treat” (ITT) population

| | B. bifidum SYN-HI-001 | |
|---|---|---|
| | (N = 221) | Placebo (N = 222) |
| | N (%) or mean ± s.d. | N (%) or mean ± s.d. |
| Age | 40.1 ± 12.8 | 42.7 ± 13.8 |
| Female Sex | 155 (70.1) | 152 (68.5) |
| Height (cm) | 172.4 ± 8.9 | 171.3 ± 9.1 |
| Weight (kg) | 73.2 ± 7.7 | 72.8 ± 16.6 |
| BMI | 24.5 ± 5.3 | 24.7 ± 5.0 |
| IBS-type | | |
| IBS-C | 54 (24.4) | 53 (23.9) |
| IBS-D | 95 (43.0) | 82 (36.9) |
| IBS-M | 14 (6.3) | 20 (9.0) |
| IBS-U | 58 (26.2) | 67 (30.2) |

1.6.3 Primary Endpoint: Composite Responder (Adequate Relief Responder 3 Combined with Pain Responder)

The primary endpoint was the composite responder, defined as a combination of 30% improvement of pain and achievement of one of the best 3 relief categories in at least 4 out of the 8 treatment weeks (50%-rule). Based on this definition, composite response was achieved in 33.5% of patients in the Bifidobacterium group (74/221) compared to 19.4% of patients in the placebo group (43/222). This is equivalent to a 1.7-fold higher success rate (95% CI: 1.3-2.4) with the study product. The Cochrane-Mantel-Haenszel test stratified by study center proved a statistical significant difference (P=0.00071). The positive result was confirmed by the per-protocol analysis. Patients treated with heat-inactivated B. bifidum SYN-HI-001 achieved 36.8% composite response compared to 19.8% when treated with placebo (P=0.0004 with the Mantel-Haenszel Test). These results are highly significant and indicate clear superiority of heat-inactivated B. bifidum SYN-HI-001 over placebo (FIG. 3). Analysis of the course of the composite responder during the two treatment intervals (week 1-4 and week 5-8) showed that the treatment with B. bifidum SYN-HI-001 was significantly superior to placebo during both the first and the second treatment interval. During weeks 1-4 31.7% of the Bifidobacteria vs. 19.8% of the placebo group (P=0.0047) and during weeks 5-8 39.4% of the Bifidobacteria vs. 29.7% (P=0.0361) fulfilled the response criteria in 50% of the time (FIG. 4).

1.6.4 Secondary Endpoints

1.6.4.1 Other Combined Responders and Adequate Relief/Symptom Relief

Secondary endpoints included a “stricter” definition of composite response: combination of 30% improvement of pain and achievement of one of the best 2 relief categories in at least 4 out of the 8 treatment weeks. Even with this “stricter” definition of adequate relief responders, this composite response was significantly higher in the Bifidobacteria group (15.8%) compared to placebo (7.7%) (P=0.0079), with the result being confirmed in the per-protocol analysis (B. bifidum SYN-HI-001: 17.9% vs. placebo: 7.5%; P=0.0031).

Analysis of the adequate relief responder alone (and not combined with another responder) results in a significant benefit for the treatment with B. bifidum SYN-HI-001. Adequate relief 3 response rate (best 3 relief categories in at least 4 out of the 8 treatment weeks) was 60.18% in the Bifidobacteria group (i.e. the verum group) and only 44.14% in the placebo group (P=0.0009). Also, adequate relief 2 response rate (best 2 relief categories in at least 4 out of the 8 treatment weeks) was significantly higher in the verum group (20.36%) compared to placebo (11.26%) (P=0.0093) (FIG. 5).

Another secondary endpoint was an improvement in symptom relief (a reduction of the mean score), which was assessed every week during the 8-weeks treatment in the patient diary. The evaluation of the symptom relief on a weekly basis showed a significant benefit for patients within the Bifidobacteria group for every single week starting from the second week of treatment (week 2) until the end of treatment (week 8). At the end of treatment, the mean score in symptom relief was 3.08 in the verum group vs. 3.44 in the placebo group (P=0.006) (FIG. 6).

1.6.4.2 Irritable Bowel Syndrome-Severity Scoring System (IBS-SSS)

IBS-SSS was assessed at baseline (visit 2), at the control visit after 4 weeks of treatment (visit 3), after the end of treatment at week 8 (visit 4) and at the end of the study at week 10 (visit 5) with a sum score ranging from 0-500. At the end of treatment (week 8), the IBS-SSS could be improved ≥50% in significantly more patients of the verum group (41.2%) as compared to patients in the placebo group (28.8%) (P=0.0072). The result was confirmed in the per-protocol analysis (B. bifidum SYN-HI-001: 37.4% vs. placebo: 25.1%; P=0.0109) (FIG. 7).

Regarding the reduction of IBS-SSS sum score from baseline to the end of treatment, the study demonstrated statistically significant superiority of B. bifidum SYN-HI-001 compared to placebo with an improvement of the sum score of −101.07 in the verum group compared to −71.24 in the placebo group (P=0.0013). Again, the result was confirmed by the per-protocol analysis (B. bifidum SYN-HI-001: −102.11 vs. placebo: −73.51; P=0.0048) (FIG. 8). Three of the subscales with each score ranging from 0-100 (bowel movement satisfaction, days with pain, and impact on daily life) showed a significant difference in favour of B. bifidum SYN-HI-001, thus strengthening evidence for the positive treatment effect in a symptom-based score. Bowel movement satisfaction was improved by −23.72 in the verum group compared to −16.62 in the placebo group (P=0.0208). Days with pain was improved by −22.71 in the verum group compared to −14.35 in the placebo group (P=0.0080). Impact on daily life was improved by −20.05 in the verum group compared to −14.15 in the placebo group (P=0.0122). The remaining two parameters of the IBS-SSS abdominal pain—severity and severity of bloating—showed a numerically greater reduction in the B. bifidum SYN-HI-001 group.

1.6.4.3 Subject's Global Assessment (SGA) of IBS Symptoms, Individual Symptoms and Composite Score 1-4

The secondary endpoints “SGA”, “abdominal pain”, “distension/bloating” and “urgency” were assessed on a daily basis in the patient diary on a 7-point Likert scale (only abdominal pain was recorded on an 11-point NRS). B. bifidum SYN-HI-001 showed a significant reduction from baseline to the end of treatment of SGA by −0.76 points vs. −0.54 points in the placebo group (P=0.0192), abdominal pain by −1.29 points vs. −0.93 points in the placebo group (P=0.0112) and distension/bloating by −0.69 points vs. −0.50 points in the placebo group (P=0.0456). A composite score 1-4 was calculated for the IBS symptoms (SGA, abdominal pain, distension/bloating and urgency). The patients in the B. bifidum SYN-HI-001 group significantly benefited from the treatment vs. placebo (change from baseline to end of treatment in verum: −1.21 points; placebo: −0.89 points; P=0.0256) (FIG. 9).

Furthermore, the symptoms “discomfort” and “pain associated with bowel movement” were assessed on a weekly basis in the patient diary on a 7-point Likert scale. Again, B. bifidum SYN-HI-001 showed a significant reduction from baseline to the end of treatment. Discomfort was reduced by −1.35 points vs. −0.92 points in the placebo group (P=0.0015) and pain associated with bowel movement by −0.88 points vs. −0.46 points in the placebo group (P=0.0231) (FIG. 10).

1.6.4.4 Health Related Quality of Life (SF-12)

Evaluation of the SF-12 sum scores showed a significant gain in quality of life in the B. bifidum SYN-HI-001 group. SF-12 sum improved from baseline to the end of treatment by 5.82 in the Bifidobacteria group and by 4.06 in the placebo group (P=0.0382). Mental health sum improved from baseline to the end of treatment by 3.31 in the Bifidobacteria group and by 1.66 in the placebo group (P=0.0309). Physical health sum was numerically larger improved in the verum group (2.51) compared to placebo (0.8965), but the difference did not reach significance. Since IBS patients are rather affected mentally than physically, this result is not surprising (FIG. 11).

1.6.4.5 Subgroup Analysis

Subgroup analysis revealed improvement of the following symptoms which are specific for corresponding IBS types:

1. Stool Frequency (IBS-C):

In IBS-C patients, the mean change from baseline to the end of treatment for frequency of bowel movements showed a significant difference in favor of B. bifidum SYN-HI-001. With B. bifidum SYN-HI-001, a mean increase of 1.66 bowel movements/week was observed in contrast to a decrease of −1.01 bowel movements/week in the placebo group (P=0.0220).

2. Loose Stool Form (IBS-D):

In the subgroup of IBS-D patients, the stool consistency, which was assessed daily in the patient diary via the BSFS (from 1=constipation to 7=diarrhoea), improved from baseline to the end of treatment significantly more in the verum group (−0.68 points) compared to the placebo group (−0.48 points) towards a harder stool consistency (P=0.0939).

3. Bowel Movement Satisfaction (IBS-M and IBS-U):

Both in the IBS-M and IBS-U subgroup, significant differences were found for bowel movement satisfaction in favor of the B. bifidum SYN-HI-001 group. At the end of treatment the difference (verum-placebo) in mean change from baseline was −27.41 [95% CI: −49.26; −5.55] in the IBS-M and −13.71 [95% CI: −24.66; −2.76] in the IBS-U subgroup.

1.6.4.6 Adverse Events

Only 15 adverse events were reported with suspected relationship to the study product, 7 in the B. bifidum SYN-HI-001 and 8 in the placebo group. No significant differences could be detected in the side effects profile of B. bifidum SYN-HI-001 vs. placebo (Table 2).

TABLE 2
Incidence of Adverse Events with suspected relationship by
preferred term (according to MedDRA):

| | B. bifidum | | | | | | |
|---|---|---|---|---|---|---|---|
| | SYN-HI-001 | | Placebo | | Total | | |
| | N | (%) | N | (%) | N | (%) | P |
| Abdominal distension | 1 | 0.45 | 1 | 0.45 | 2 | 0.45 | 10.000 |
| Abdomina pain | 2 | 0.90 | 1 | 0.45 | 3 | 0.68 | 0.6233 |
| Abdominal pain upper | 0 | 0.00 | 1 | 0.45 | 1 | 0.23 | 10.000 |
| Constipation | 0 | 0.00 | 1 | 0.45 | 1 | 0.23 | 10.000 |
| Diarrhoea | 0 | 0.00 | 2 | 0.90 | 2 | 0.45 | 0.4989 |
| Gastroenteritis | 1 | 0.45 | 0 | 0.00 | 1 | 0.23 | 0.4989 |
| Irritable bowel | 1 | 0.45 | 0 | 0.00 | 1 | 0.23 | 0.4989 |
| syndrome aggravated | | | | | | | |
| Liver function test | 0 | 0.00 | 2 | 0.90 | 2 | 0.45 | 0.4989 |
| Nausea | 1 | 0.45 | 0 | 0.00 | 1 | 0.23 | 0.4989 |
| Urinary incontinence | 1 | 0.45 | 0 | 0.00 | 1 | 0.23 | 0.4989 |

Most adverse events were mild to moderate in severity and resolved after symptomatic treatment. Most of the events were related to gastrointestinal symptoms and can be considered concomitant symptoms of the underlying disease.

Only two patients reported serious adverse events: acute coronary syndrome and femoral neck fracture, which were both rated as not treatment related and which both occurred in the placebo group. No deaths were reported in this study.

Medications taken for IBS symptoms were not significantly different between the two treatment groups, as were discontinuations of study treatment. Also laboratory values are not indicative for any safety risk by B. bifidum SYN-HI-001. The evaluation of vital signs and physical examination revealed nothing unusual. Global assessment of tolerability at the end of treatment was rated very good or good in 90.5% of the patients in the B. bifidum SYN-HI-001 and 86.0% in the placebo group (88.3% of the total study population; difference is not significant).

Overall the study demonstrated good tolerability and did not indicate any safety risk for the use of heat-inactivated B. bifidum SYN-HI-001 in IBS patients.

The study provided herein shows for the first time that non-viable bacteria of the strain B. bifidum SYN-HI-001 possess in vivo efficacy suitable for the treatment of IBS. Different from probiotic compositions, i.e. compositions based on viable bacteria, these non-viable bacteria of the present invention additionally provide a reduced risk of being associated with adverse side effects, which have previously been reported e.g. in Besselink et al., 2008 for seriously ill subjects. Thus, the non-viable bacteria of the present invention, as well as fragments thereof, provide a promising and safe new tool for the treatment of gastrointestinal disorders.

### FURTHER REFERENCES

Alving C R. (1993). Lipopolysaccharide, lipid A, and liposomes containing lipid A as immunologic adjuvants. Immunobiology, 187(3-5), 430-446.
Bausserman, M., & Michail, S. (2005). The use of Lactobacillus GG in irritable bowel syndrome in children: a double-blind randomized control trial. The Journal of pediatrics, 147(2), 197-201.
Besselink, M. G., et al., Probiotic prophylaxis in predicted severe acute pancreatitis: a randomised, double-blind, placebo-controlled trial. Lancet, 2008. 371(9613): p. 651-9.
Brenner, D. M., & Chey, W. D. (2009). Bifidobacterium infantis 35624: A novel probiotic for the treatment of irritable bowel syndrome. Reviews in gastroenterological disorders, 9(1), 7-15.
Brul, Stanley, Suzanne Van Gerwen, and Marcel Zwietering, eds. Modelling microorganisms in food. Elsevier, 2007.
Camilleri, M., & Ford, A. C. (2017). Pharmacotherapy for Irritable Bowel Syndrome. Journal of Clinical Medicine, 6(11), 101.
Chang, L. “Epidemiology and quality of life in functional gastrointestinal disorders.” Alimentary pharmacology & therapeutics 20.s7 (2004): 31-39.
Ducrottè, P., Sawant, P., & Jayanthi, V. (2012). Clinical trial: Lactobacillus plantarum 299v (DSM 9843) improves symptoms of irritable bowel syndrome. World journal of gastroenterology: WJG, 18(30), 4012.
e Silva, J. Paulo Sousa, and Ana Cristina Freitas, eds. Probiotic bacteria: fundamentals, therapy, and technological aspects. Crc Press, 2014.
Eun C S, Kim Y S, Han D S, Choi, JH, Lee A R, Park Y K. Lactobacillus casei prevents impaired barrier function in intestinal epithelial cells. APMIS 2011; 119: 49-56.
Farup, P. G., Jacobsen, M., Ligaarden, S. C., & Rudi, K. (2012). Probiotics, symptoms, and gut microbiota: what are the relations? A randomized controlled trial in subjects with irritable bowel syndrome. Gastroenterology research and practice, 2012.
Gargon & Van Mechelen. (2011). Recent clinical experience with vaccines using MPL- and QS-21-containing adjuvant systems. Expert Rev Vaccines, 10(4), 471-486.
Guideline on the evaluation of medicinal products for the treatment of irritable bowel syndrome. EMA/CHMP/60337/2013.
Isolauri, E., Rautanen, T., Juntunen, M., Sillanaukee, P., & Koivula, T. (1991). A human Lactobacillus strain (Lactobacillus casei sp strain GG) promotes recovery from acute diarrhea in children. Pediatrics, 88(1), 90-97.
Kim, H. J., Vazquez Roque, M. I., Camilleri, M., Stephens, D., Burton, D. D., Baxter, K., & Zinsmeister, A. R. (2005). A randomized controlled trial of a probiotic combination VSL #3 and placebo in irritable bowel syndrome with bloating. Neurogastroenterology & Motility, 17(5), 687-696.
Kneifel, Wolfgang, and Seppo Salminen, eds. Probiotics and health claims. John Wiley & Sons, 2010.
Layer, P., et al. “S3-Leitlinie Reizdarmsyndrom: Definition, Pathophysiologie, Diagnostik und Therapie. Gemeinsame Leitlinie der Deutschen Gesellschaft for Verdauungs-und Stoffwechselkrankheiten (DGVS) und der Deutschen Gesellschaft für Neurogastroenterologie und MotilitAt (DGNM).” Zeitschrift für Gastroenterologie 49.02 (2011): 237-293.
Lopetuso, L. R., Scaldaferri, F. R. A. N. C. O., Bruno, G., Petito, V., Franceschi, F., & Gasbarrini, A. (2015). The therapeutic management of gut barrier leaking: the emerging role for mucosal barrier protectors. Eur Rev Med Pharmacol Sci, 19(6), 1068-1076.
Mitra, A. K., & Rabbani, G. H. (1990). A double-blind, controlled trial of bioflorin (Streptococcus faecium SF68) in adults with acute diarrhea due to Vibrio cholerae and enterotoxigenic Escherichia coli. Gastroenterology, 99(4), 1149-1152.
Niv, E., Naftali, T., Hallak, R., & Vaisman, N. (2005). The efficacy of Lactobacillus reuteri ATCC 55730 in the treatment of patients with irritable bowel syndrome—a double blind, placebo-controlled, randomized study. Clinical Nutrition, 24(6), 925-931.
Petrovsky & Aguilar. (2004). Vaccine adjuvants: current state and future trends. Immunol Cell Biol, 82(5), 488-496.
Piche, T., et al., Impaired intestinal barrier integrity in the colon of patients with irritable bowel syndrome: involvement of soluble mediators. Gut, 2009. 58(2): p. 196-201.
Resta-Lenert, S., & Barrett, K. E. (2006). Probiotics and commensals reverse TNF-α- and IFN-γ-induced dysfunction in human intestinal epithelial cells. Gastroenterology, 130(3), 731-746.
Rome, F., Guidelines—Rome III Diagnostic Criteria for Functional Gastrointestinal Disorders. J Gastrointestin Liver Dis, 2006. 15(3): p. 307-12.
Sen, S., Mullan, M. M., Parker, T. J., Woolner, J. T., Tarry, S. A., & Hunter, J. O. (2002). Effect of Lactobacillus plantarum 299v on colonic fermentation and symptoms of irritable bowel syndrome. Digestive diseases and sciences, 47(11), 2615-2620.
Steiner et al. (1960). The local and systemic effects of Freund's adjuvant and its fractions. Archives of Pathology, 70, 424-434.
Tsuchiya, J., Barreto, R., Okura, R., Kawakita, S., Fesce, E., & Marotta, F. (2004). Single-blind follow-up study on the effectiveness of a symbiotic preparation in irritable bowel syndrome. Journal of Digestive Diseases, 5(4), 169-174.
Vivinus-Nébot, M., et al. “Combination of allergic factors can worsen diarrheic irritable bowel syndrome: role of barrier defects and mast cells.” The American journal of gastroenterology 107.1 (2012): 75.
Weiner et al. (1997). Immunostimulatory oligodeoxynucleotides containing the CpG motif are effective as immune adjuvants in tumor antigen immunization. PNAS, 94(20), 10833-10837.
Yoo et al. (1998). Adjuvant activity of muramyl dipeptide derivatives to enhance immunogenicity of a hantavirus-inactivated vaccine. Vaccine, 16(2-3), 216-224.

1-3. (canceled)

4. A composition comprising, as an active ingredient, non-viable bacteria of the Bifidobacterium bifidum strain SYN-HI-001 deposited under deposit No. DSM 24514, or one or more fragments thereof.

5. (canceled)

6. The composition of claim 4, wherein the composition is a pharmaceutical composition or a food composition.

7. The composition of claim 4, wherein the composition comprises, as an active ingredient, non-viable bacteria of the Bifidobacterium bifidum strain SYN-HI-001 deposited under deposit No. DSM 24514.

8. A method of preparing non-viable bacteria of the Bifidobacterium bifidum strain SYN-HI-001 deposited under deposit No. DSM 24514, or one or more fragments thereof, the method comprising:
(a) providing bacteria of the Bifidobacterium bifidum strain deposited under deposit No. DSM 24514; and
(b) inactivating the bacteria provided in step (a) to obtain non-viable bacteria of the Bifidobacterium bifidum strain SYN-HI-001.

9. The method of claim 8, wherein the bacteria are inactivated in step (b) by subjecting the bacteria to heat, pressure, sonication, irradiation, drying, pulsed electric field (PEF), supercritical CO₂ and/or a pH change.

10.-11. (canceled)

12. A method of treating a gastrointestinal disorder, the method comprising administering non-viable bacteria, or one or more fragments thereof, obtainable by the method of claim 8 to a subject in need thereof.

13. (canceled)

14. A method of treating a gastrointestinal disorder, the method comprising administering non-viable bacteria of the Bifidobacterium bifidum strain SYN-HI-001 deposited under deposit No. DSM 24514, or one or more fragments thereof, to a subject in need thereof.

15. (canceled)

16. The method of claim 14, wherein the gastrointestinal disorder is selected from the group consisting of irritable bowel syndrome (IBS), inflammatory bowel disease (IBD), Crohn's disease, ulcerative colitis, pouchitis, post infection colitis, diarrhoea, constipation, dyspepsia and/or associated dyspeptic symptoms, gastroparesis, and intestinal pseudo-obstruction.

17. The method of claim 14, wherein the non-viable bacteria are administered orally in a daily amount of at least about 10² non-viable cells.

18. The method of claim 14, wherein the non-viable bacteria, or the one or more fragments thereof, are administered to a human subject.

19. The method of claim 14, wherein the non-viable bacteria, or the one or more fragments thereof, are administered to an immuno-compromised subject.

20. The method of claim 14, wherein the method comprises administering non-viable bacteria of the Bifidobacterium bifidum strain SYN-HI-001 deposited under deposit No. DSM 24514 to the subject.
